(19) United States
(12) Patent Application Publication
(10) Pub. No.: US 2008/0069886 A1
(43) Pub. Date: Mar. 20, 2008

(54) Spironolactone nanoparticles, compositions and methods related thereto

(75) Inventors: Guy VERGNAULT, Kembs Loechle (FR); Pascal GRENIER, Kappelen (FR); Alain NHAMIAS, Bartenheim (FR)

Correspondence Address: MINTZ, LEVIN, COHN, FERRIS, GLOVSKY;AND POPEO, P.C., ONE FINANCIAL CENTER, BOSTON, MA, 02111, US

(73) Assignee: JAGOTEC AG, Muttenz (CH)

(21) Appl. No.: 11/881,337

(22) Filed: Jul. 25, 2007

Related U.S. Application Data
(63) Continuation of application No. 10/480,573, filed on Dec. 12, 2003, now abandoned.

(30) Foreign Application Priority Data
Jun. 14, 2001 (GB) 0114532.5

(51) Int. Cl. A61K 31/58; A61K 9/14; A61P 9/04

(52) U.S. Cl. 424/489; 514/173

(57) ABSTRACT

This invention relates to nanoparticles comprising spironolactone. The nanoparticles have a mean diameter, measured by photon spectroscopy, in the range of from about 300 nm to about 900 nm.

### FIELD OF INVENTION

The present invention relates to the drug substance spironolactone in the form of nanoparticles, to methods of preparing said nanoparticles, formulations containing said nanoparticles, and the use of said nanoparticulate drug substance. In particular the present invention relates to nanosuspensions comprising spironolactone.

### BACKGROUND OF INVENTION

Spironolactone is known as an aldosterone inhibitor having utility as a potassium sparing diuretic. It is commercially available as e.g. aldactone and may be employed e.g. in the treatment of congestive heart failure. Spironolactone has extremely low solubility in water, viz: 2.8 mg/100 ml This can adversely affect absorption of the drug substance in vivo, leading to poor bioavailability. Consequently higher amounts of the drug substance are required to achieve the desired blood levels. The poor solubility of spironolactone also restricts the options available for formulating the drug substance.

Following oral administration, the absorption of drugs from the intestine is mainly dependent on their solubility in the intestinal fluids and their intestinal permeability. Poorly soluble drugs generally have low dissolution rates and exhibit only a small concentration gradient across the intestinal mucosa, which can result in low and unreliable levels of absorption. Drug substances which have low solubility also suffer from disadvantages in respect of other routes of administration, for example, by injection. Thus, it may only be possible to achieve very dilute solutions which do not provide the required dosage. In such circumstances it may be necessary to administer the drug as a continuous infusion rather than as a bolus injection. In some cases it may not be possible to achieve formulations suitable for parenteral administration at all.

Significant efforts have been directed to producing drug substances in the form of microparticles and nanoparticles. However, preparation of such small particles is not a trivial matter and can give rise to further difficulties both in relation to technical aspects of the process and in obtaining a satisfactory product. Thus for example there can be difficulties, especially on a manufacturing scale in obtaining a consistent and narrow particle size range. Furthermore, it is necessary to obtain stable products, e.g. nanosuspensions, but microparticles and nanoparticles have a tendency to aggregate and flocculate, which has adverse consequences for the stability of the product. A number of different approaches have been investigated for the preparation of microparticles and nanoparticles.

U.S. Pat. No. 5,091,188 describes a method for preparing injectable solutions of water-insoluble drugs, which comprises reducing the crystalline drug substance to dimensions in the range 50 nm to 10 μm, by sonication or other processes inducing high shear, in the presence of a phospholipid or other membrane-forming amphipathic lipid, whereby the drug microcrystals become coated with said lipid.

U.S. Pat. No. 5,145,684 describes particles of crystalline drug substance having a non-cross linked surface modifier adsorbed on the surface and an effective average particle size of less than about 40 nm. These particles are said to be prepared by milling in the presence of grinding media, using for example a ball mill, an attrition mill, a vibratory mill or a media mill.

International Patent Application WO 96/14830 (U.S. Pat. No. 5,858,410) describes a drug carrier which comprises particles of a pure active compound which is insoluble or only sparingly soluble in water, which has an average diameter of 10 nm to 1,000 nm and the proportion of particles larger than 5 .mu.m in the total population is less than 0.1%. Preparation of the particles, with or preferably without surfactant, by means of cavitation (e.g. using a piston-gap homogenizer) or by shearing and impact forces (i.e. the jet stream principle) is also described.

### SUMMARY OF INVENTION

We have now found that spironolactone can advantageously be prepared in nanoparticulate form, said nanoparticles being obtained in a consistent and narrow particle size range. Advantageously, nanoparticulate spironolactone is provided in the form of a nanosuspension. We have further surprisingly found that said nanosuspension has increased flux across the intestinal membrane and an improved pharmacokinetic profile following oral administration to rats.

### BRIEF DESCRIPTION OF THE DRAWINGS

FIG. 1 is a bar graph illustrating the spironolactone fluxes at various dilutions.

FIGS. 2A and 2B are line graphs detailing the pharmacokinetic profiles of the spironolactone nanosuspension over time.

FIG. 3 is a line graph of the spironolactone nanosuspensions fasted conditions—6 dogs over time.

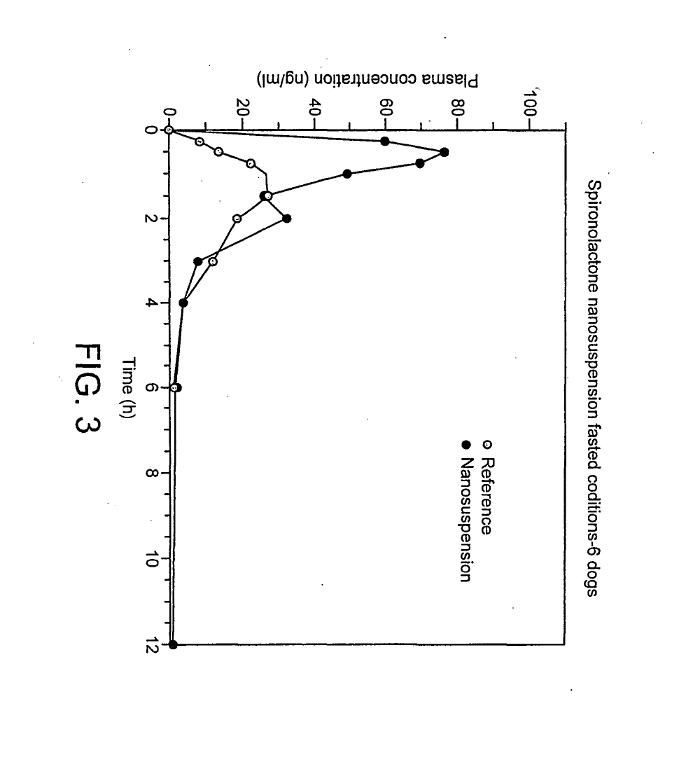

FIGS. 4A and 4B show a comparison study of spironolactone nanosuspensions fed/fasted in 6 dogs over time.

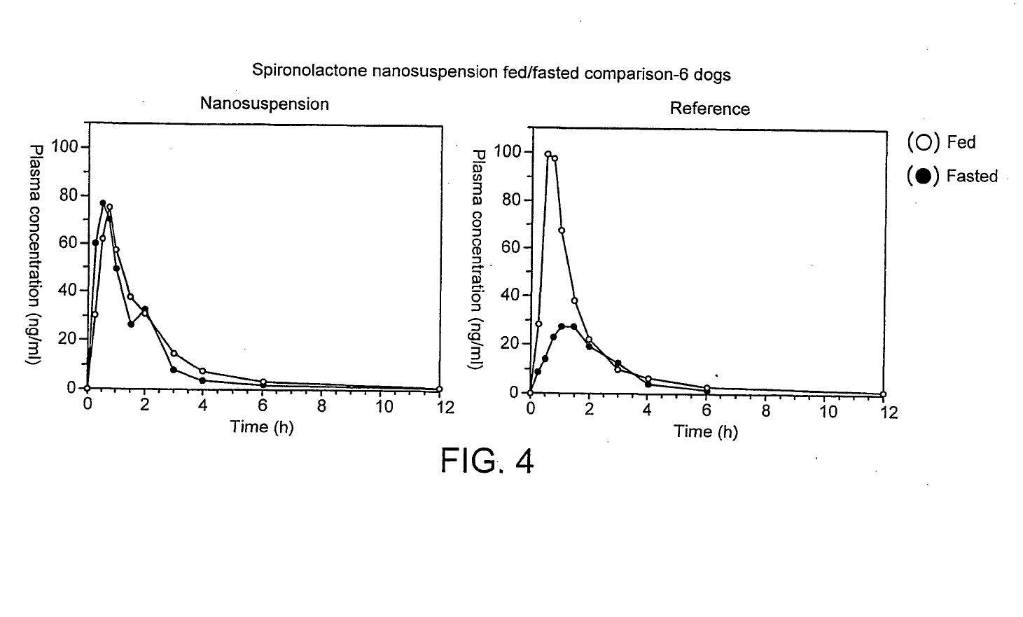

### DETAILED DESCRIPTION

In a first aspect therefore the present invention provides nanoparticles comprising spironolactone, said nanoparticles having a mean diameter, measured by photon correlation spectroscopy, in the range of from about 300 nm to about 900 nm, preferably 400 nm to 600 nm.

As is well known in the pharmaceutical art, particle size may be measured by a variety of methods, which can give rise to apparently different reported particle sizes. Such methods include photon correlation spectroscopy (PCS) and laser diffraction. Furthermore the particle size may be reported as an average particle size (e.g. a number average, weight average or volume average particle size). In the present specification, unless indicated otherwise, the particle size will be quoted as a volume average particle size. Thus for example, a D₅₀ of 500 nm indicates that 50% by volume of the particles have a diameter of less than 500 nm. Alternatively it can be stated that the particles having a diameter of less than 500 nm occupy 50% of the total volume occupied by the total number of particles.

When the particle size of spironolactone according to the present invention is measured by laser diffraction the D₅₀ is in the range 350-750 nm and the D₉₉ is in the range 500-900 nm.

Nanosuspensions and nanoparticles comprising spironolactone according to the present invention preferably incorporate a stabiliser to prevent aggregation of the nanoparticles. Such stabilisers, which are well known in the art, are described in more detail hereinafter.

In this specification nanoparticles comprising spironolactone and nanosuspensions comprising spironolactone according to the present invention will be referred to as nanoparticulate spironolactone. It should be appreciated that this term also includes nanoparticles and nanosuspensions comprising spironolactone in association with a stabiliser.

Nanoparticulate spironolactone according to the invention, may be prepared by any known method for the preparation of nanoparticles, in particular by cavitation.

In a second aspect the present invention provides a process for preparing nanoparticles comprising spironolactone which comprises subjecting a coarse dispersion of spironolactone to cavitation. Preferably the nanoparticles are prepared using a high pressure piston-gap homogeniser. The nanoparticles may be associated with a stabiliser. Such stabilisers, which are well known in the art, are described in more detail hereinafter.

For the preparation of nanoparticles it is preferred that the spironolactone starting material be utilised in the form of coarse particles, preferably having a particle size of less than about 100 μm. If necessary, the particle size of the spironolactone may be reduced to this level by conventional means, such as milling. The coarse particles of spironolactone are preferably dispersed in a liquid medium comprising a solvent in which the drug substance is essentially insoluble. In the case of spironolactone the liquid medium preferably comprises an aqueous solvent and most preferably consists essentially of water. The concentration of spironolactone in the said dispersion of coarse particles may be in the range 0.1 to 50%. The coarse dispersion may then be utilised in any known method for obtaining nanoparticles.

A preferred method is high pressure homogenization, wherein particle size is reduced mainly by cavitation. This is most preferably achieved using a high pressure piston-gap homogeniser. In this method, the dispersion of coarse particles is forced at a high flow rate through a gap which is approximately 25 μm wide. The static pressure exerted on the liquid falls below the vapour pressure of the liquid. The liquid therefore boils, resulting in the formation of gas bubbles within the area of the gap. However, once the liquid exits from the gap, normal pressure prevails and the gas bubbles collapse. The powerful implosion forces which result are strong enough to break up the coarse particles of drug substance, resulting in the formation of nanoparticles.

High pressure homogenisation may be carried out at a pressure in the range 100 to 3000 bar, preferably 1000 to 2000 bar (10⁷ to 3×10⁸ Pa, preferably 10⁸ to 2×10⁸ Pa) and at a temperature in the range 0 to 50° C., preferably 10 to 20° C., eg around 15° C. The homogenisation may be carried out in a series of cycles until the desired particle size is obtained, or as a continuous process, e.g. over a period of 2-30 hours, preferably 2-10 hours.

Nanosuspensions of spironolactone according to the present invention preferably incorporate a stabiliser to prevent aggregation of the nanoparticles. Said stabiliser may be introduced at any suitable stage during the manufacture of the nanosuspension. Thus for example, surfactant may be added to the initial coarse dispersion prior to the formation of nanoparticles or after reduction of the particles size, e.g. by high pressure homogenization, has taken place. Alternatively a portion of the stabiliser may be added before and a portion after the step of particle size reduction. Preferably stabiliser is present in the coarse dispersion. The concentration of stabiliser, either in the coarse dispersion or the nanosuspension may be in the range 0 to 10%.

Stabilisers which may be employed in the preparation of nanosuspensions according to the present invention may be selected from conventional stabilisers, and may include compounds which are also described as surfactants and surface modifiers. Thus examples of stabiliser which may be employed include:
polyoxyethylene sorbitan fatty acid esters, e.g. Tweens and Spans;
polyoxyethylene stearates; polyoxyethylene alkyl esters; polyethylene glycols;
block polymers and block copolymers such as poloxamers e.g Lutrol F68, and poloxamines; lecithins of various origin (e.g. egg-lecithin or soya-lecithin), chemically-modified lecithins (e.g. hydrated lecithin), as well as phospholipids and sphingolipids, sterols (e.g. cholesterin derivatives, as well as stigmasterin), esters and ethers of sugars or sugar alcohols with fatty acids or fatty alcohols (e.g. saccharose monostearate);
ethoxylated mono- and diglycerides, ethoxylated lipids and lipoids, dicetyl phosphate, phosphatidyl glycerine, sodium cholate, sodium glycolcholate, sodium taurocholate; sodium citrate;
cellulose ethers and cellulose esters (e.g. methyl cellulose, hydroxyethyl cellulose, hydroxypropyl cellulose, sodium carboxymethyl cellulose), polyvinyl derivatives such as polyvinyl alcohol, polyvinyl pyrrolidone, polyvinyl acetate, alginates, polyacrylates (e.g. carbopol), xanthanes; pectins, gelatin, casein, gum acacia, cholesterol, tragacanth, stearic acid, calcium stearate, glyceryl monostearate, dioctyl sodium sulfosuccinate (sodium docusate); sodium lauryl sulfate, sodium dodecyl sulphate, benzalkonium chloride, alkyl aryl polyether sulfonate, polyethylene glycols;
colloidal silicon dioxide, magnesium aluminum silicate; and phosphates.

A preferred stabiliser is sodium docusate, which is commercially available as a solution in propylene glycol, under the name Octowet 70™.

It will be appreciated from the foregoing that the process is carried out in a liquid medium and hence the nanoparticulate spironolactone product is initially obtained in the form of a nanosuspension. If desired the liquid medium may be removed, e.g. by lyophilisation or spray drying to provide nanoparticulate spironolactone in solid form. It will be appreciated that where a stabiliser is present during the manufacture of a nanosuspension, the corresponding dried nanoparticulate product will be associated with said stabiliser.

The spironolactone nanosuspensions and nanoparticles according to the present invention may be formulated for pharmaceutical use, optionally using pharmaceutically acceptable excipients and carriers well known in the art. They may be administered as a medicament by any convenient route, eg by parenteral, oral, topical, buccal, sublingual, nasal, pulmonary, rectal or transdermal administration.

In a third aspect therefore the invention provides a pharmaceutical formulation comprising nanoparticles comprising spironolactone, said nanoparticles having a mean diameter, measured by photon correlation spectroscopy, in the range of from about 300 nm to about 900 nm, preferably 400 nm to 600 nm. Pharmaceutical formulations according to the present invention advantageously comprise a nanosuspension, most preferably in aqueous solution. Pharmaceutical formulations according to the present invention may be prepared according to methods well known in the art.

Thus for example, solid dosage forms, eg for oral administration may be prepared by spray-coating the nanosuspension comprising spironolactone on to a sugar sphere or other suitable solid pharmaceutical excipient.

Dosage forms for pulmonary administration by inhalation may be provided as an aerosol, comprising an aqueous nanosuspension of spironolactone. A dry powder for inhalation may be prepared by spraying the aqueous dispersion on to carrier particles, such as lactose.

Spironolactone formulations according to the present invention may be used for the treatment of congestive heart failure and other conditions which may be treated with an aldosterone inhibitor.

In a further aspect the present invention provides the use of nanoparticulate spironolactone in the treatment of a condition known to be treatable with an aldosterone inhibitor, e.g. congestive heart failure.

Experimental

Table I illustrates representative preparations of spironolactone according to the present invention.

Preparation of Nanosuspensions

A preparation of an aqueous solution of the stabiliser was incorporated into water or buffer for injection under magnetic stirring until a clear solution was obtained. A slurry was formed by wetting the spironolactone with the appropriate quantity of the aqueous solution of the surfactant. The resulting suspension was dispersed using a high shear-dispersing instrument. The suspensions were left under magnetic agitation to eliminate foaming. The resulting suspensions were passed through a high-pressure piston gap homogenizer to obtain a nanosuspension. Formulations 1-7 were prepared using an Avestin C5™ and Formulations 8 and 9 were prepared using an Avestin C50™. During homogenization the drug particles are disrupted due to cavitation effects and shear forces to form small micro- and nanoparticles. The particle sizes were determined by photon correlation spectroscopy (PCS) using a Zetasizer 3000 HS™ (Malvern). D₅₀ and D₉₀ were measured by laser diffraction using a Coulter LS230.

TABLE I

| | Formulation | | | | | | | | |
|---|---|---|---|---|---|---|---|---|---|
| | 1 | 2 | 3 | 4 | 5 | 6 | 7 | 8 | 9 |
| Spironolactone % | 10 | 10 | 20 | 10 | 10 | 10 | 10 | 10 | 10 |
| Sodium lauryl | 1 | — | — | 0.1 | 0.4 | 0.1 | — | — | — |
| sulphate % | | | | | | | | | |
| Lutrol F68 % | — | 1 | 1 | 0.4 | 0.1 | 0.4 | — | — | — |
| NaCl | — | — | — | — | — | 0.9 | — | — | — |
| Octowet 70 | — | — | — | — | — | — | 0.5 | 0.5 | 0.5 |
| (sodium | | | | | | | (0.35) | (0.35) | (0.35) |
| docusate) % | | | | | | | | | |
| Water | QS to 100% | | | | | | | | |
| Process | | | | | | | | | |
| Conditions | | | | | | | | | |
| Pressure (bars) | 1500 | 1500 | 1500 | 1500 | 1500 | 1500 | 1500 | 1500 | 1500 |
| Time (min) | 90 | 180 | 180 | | 180 | 180 | 300 | 300 | 1080 |
| Sample volume | 40 | 40 | 40 | 40 | 40 | 40 | 40 | 100 | 500 |
| (ml) | | | | | | | | | |
| Results | | | | | | | | | |
| D₅₀ (micron) | 1.69 | 0.85 | 1.06 | 0.84 | 0.88 | 0.86 | 0.78 | 0.54 | 0.539 |
| D₉₀ (micron) | 4.39 | 1.83 | 2.49 | 1.92 | 1.82 | 1.5 | 1.8 | 0.68 | 0.772 |
| PCS mean | — | 581 | 880 | 608 | 681 | 656 | 609 | 415 | 436 |
| diameter | | | | | | | | | |
| PI | — | 0.7 | 0.2 | 0.15 | 0.03 | 0.1 | 0.2 | 0.05 | 0.1 |

Biological Test Results

Nanosuspensions of spironolactones according to the present invention were tested to study the effect of the different saturation concentrations provided by the formulations on the drug transport across Caco-2 cell monolayers.

The formulation used in this study was Formulation 8 shown in Table 1 hereinbefore.

Preparation of Test Solutions

The nanosuspensions were diluted with different volumes of Hanks Balanced Salt Solution (HBSS) supplemented with 25 mM MES adjusted to pH 6.5 and shaken until an equilibrium was reached. For the reference solutions an excess amount of coarse powder of each drug was shaken in HBSS/MES in the presence of the corresponding surfactant concentration until the saturation concentration was reached. Separation of the solution from the sediment was performed by centrifugation for 15 min at 4500 ref.

Absorption Studies

Caco-2 cells (passage 33-41) were cultured for 21-27 days on 24 mm polycarbonatE filter membranes (0.4 μm pore size; Transwell, Coming, Mass.). 2.5 ml of test solution was added to the apical and 2.5 ml buffer to the basolateral side. Samples from the receiver chamber were collected at 0, 30, 60, 90, 120 min and volume was replaced by fresh medium. Samples were analysed for the radiolabelled marker molecules by liquid scintillation counting and for the spironolactone by HPLC. As integrity markers, ¹⁴C-mannitol and ³H-metoprolol were used. In addition TEER (transepithelial electrical resistance) measurements at the beginning and the end of each experiment were conducted.

The fluxes of drug were calculated from the slope of the amounts of drug transported across the monolayer versus time.

Results

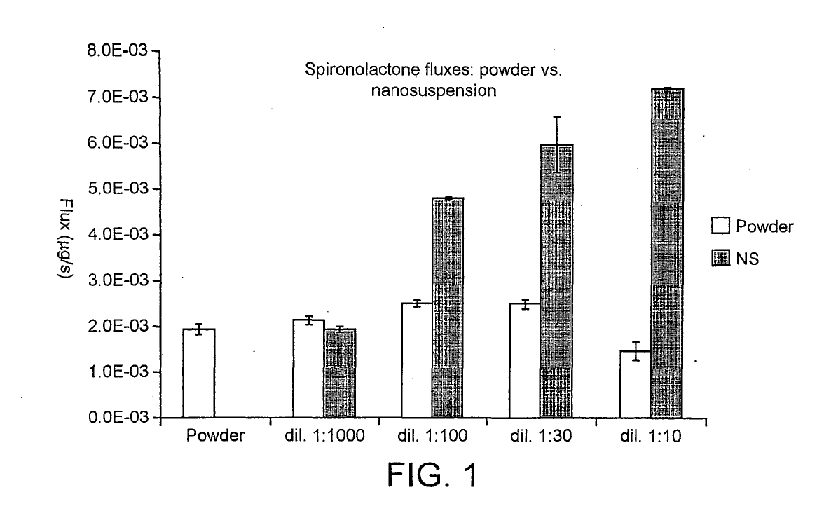

FIG. 1 illustrates the steady-state fluxes across the intestinal membrane for spironolactone. At dilutions of 1:100, 1:30 and 1:10, the flux values were higher for the diluted nanosuspension as donor solution as compared to the coarse suspension.

Oral Absorption Studies

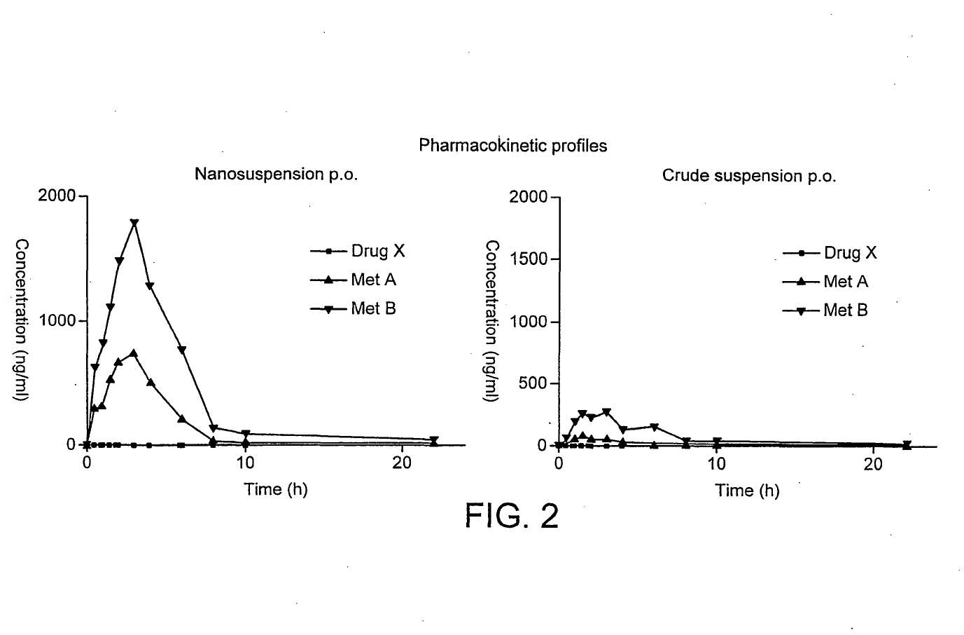

Following oral administration to rats, spironolactone nanosuspension according to the present invention gave significantly higher plasma levels of drug metabolites than a corresponding coarse suspension, as shown by FIG. 2.

What I claim is:

1. A nanosuspension of spironolactone nanoparticles having a mean diameter in the range of from about 300 nm to about 900 nm, wherein the spironolactone nanoparticles are prepared by subjecting a coarse dispersion of spironolactone to cavitation.

2. The nanosuspension of claim 1, wherein the nanoparticles have a mean diameter in the range of from about 400 nm to about 600 nm.

3. The nanosuspension of claim 1 which is an aqueous nanosuspension.

4. The nanosuspension of claim 1, further comprising a stabiliser.

5. The nanosuspension of claim 4, wherein the stabiliser is sodium docusate.

6. The nanosuspension of claim 1, wherein the cavitation is effected using a high-pressure piston-gap homogenizer.

7. A pharmaceutical formulation comprising the nanosuspension of claim 1.

8. A method of treating a subject with a condition requiring treatment with an aldosterone inhibition, the method comprising administering to the subject the nanosuspension of claim 1 in an amount effective to treat the condition.

9. The method of claim 8, wherein the condition is congestive heart failure.
